(19) United States
(12) Patent Application Publication
(10) Pub. No.: US 2023/0071163 A1
(43) Pub. Date: Mar. 9, 2023

(54) FUNCTIONAL FOOD COMPOSITION COMPRISING COLLAGEN AND METHODS TO PRODUCE THEREOF

(71) Applicant: SOCIETE DES PRODUITS NESTLE S.A., Vevey (CH)

(72) Inventors: KURT SEIDENSTICKER, Chicago, IL (US); KATIE COTE, Chicago, IL (US); CRISTINA CUTRI, Chicago, IL (US); COREY FRIESE, Chicago, IL (US)

(21) Appl. No.: 17/759,330

(22) PCT Filed: Jan. 27, 2021
(86) PCT No.: PCT/EP2021/051793

§ 371 Date: Jul. 22, 2022

Related U.S. Application Data
(60) Provisional application No. 62/966,325, filed on Jan. 27, 2020.

(51) Int. Cl. A23L 33/18; A23L 33/185; A23P 10/28

(52) U.S. Cl. CPC A23L 33/18; A23L 33/185; A23P 10/28; A23V 2002/00

(57) ABSTRACT

The present invention is directed to functional food compositions comprising collagen. In one embodiment, the invention relates to a collagen and nut protein blend, wherein the composition is a solid or semi-solid that is shelf-stable and has a soft texture. The present invention also includes compositions with other nut protein blends, and compositions that further do not contain gluten. A method of making the compositions of the present invention is also claimed.

### FIELD OF THE INVENTION

The present invention relates to a functional food composition comprising collagen. In one embodiment, the invention relates to a collagen and nut protein blend, wherein the composition is a solid or semi-solid that is shelf-stable and has a soft texture. The present invention also includes compositions with other nut protein blends, and compositions that further do not contain gluten. A method of making the compositions of the present invention is also described.

### BACKGROUND OF THE INVENTION

Connective tissue is a major part of muscles, tendons, ligaments, cartilage, and bones. It is the second biggest mass and organ of the body and is important for physical performance. Connective tissue contains mainly collagen structures, which provides bones, tendons, and ligaments structure. Connective tissue contains a matrix of very flexible collagen strings, which is highly stabilized by cross-linking the gelatin-like strings of collagen with pyridinoline (PD) and deoxypyrolidine (DPD). PD and DPD are present only in mature collagen and not in freshly built collagen. In the process of making collagen, collagen strings are first built and then cross-linked with PD and DPD.

The development of collagen enhanced compositions that are solids or semi-solid products also has encountered obstacles in formulation. Products containing collagen harden quickly over time, creating shelf-life difficulties as well as creating foodstuff that is hard to chew, negatively effecting the eating experience. Hardened products can make chewing and swallowing difficult, tiring, or uncomfortable, problems that can be compounded by dehydration after physical activity. The manufacturing of collagen containing bars further poses a challenge given that the process of physically forming the bars increases the hardening process and toughness of the resulting product.

A second problem encountered in developing collagen-containing edible products involves taste and mouth-feel. When used in sports performance and nutrition, it is desirable to not use sugar-containing ingredients and/or ingredients with gluten, which is linked to gastrointestinal distress and inflammation. Sugar-added ingredients and gluten ingredients, however, are commonly used for binders in the formation of food bars and finding suitable substitutes can be difficult.

Developing a shelf-stable and soft textured collagen-containing functional food composition without gluten is desired.

### SUMMARY OF THE INVENTION

The present invention is directed to a shelf-stable and soft textured functional food composition comprising collagen and almond protein blend. In one embodiment, the collagen is present in an amount of about 15 to 25 weight percent of the composition and the almond protein blend is present in an amount of about 25 to 40 weight percent of the composition. In another embodiment, the amount of collagen present in a 50 g serving is about 7-13 g and the amount of almond protein blend is about 12-20 g per 50 g serving. In one embodiment, the amount of collagen present is about 10 g per 50 g serving and the amount of almond protein blend is about 16-18 g per 50 g serving. In another embodiment, protein blends from nuts other than almonds are used.

The present invention is further directed to a shelf-stable and soft textured functional food composition comprising collagen peptides and almond protein blend that does not include ingredients containing gluten. In one embodiment, alternative forms of sweeteners are added to the composition that not well metabolized by the body, do not cause a high glycemic index response in the blood, and/or do not require labeling as an added sugar on a nutritional label. Such non-sugar sweeteners can be artificial or natural products, including but not limited to stevia, luo han guo (monk) fruit extract, sucralose, aspartame, acesulfame potassium, neotame, saccharin, advantame, allulose, and/or sugar alcohols extracted from fruit, vegetables, or those that are man-made may be added to the composition of the present invention, including but not limited to erythritol.

The composition of the present invention may be in the form of a solid or semi-solid foodstuff, including but not limited to a bar, bites, cracker, or cookie. The present invention also includes the formation of bars, crackers, and cookies using cold slab manufacturing techniques known in the art and suitable pressure.

### DETAILED DESCRIPTION OF THE INVENTION

The present invention relates to a functional food composition that may be consumed, for example as a meal or as a meal supplement, to maintain collagen levels in the body, which help to support muscles, joints, tendons, and ligaments. The compositions of the present invention comprise collagen, preferably in the form of collagen peptides, in combination with almond protein blend or other nut protein blends.

Collagen is present in the composition in an amount of about 15 to 25 weight percent of the composition, about 15 to 20 weight percent of the composition, about 17 to 22 weigh percent of the composition, or about 20 to 25 weight percent of the composition. In another embodiment, the amount of collagen present in a 50 g serving is about 7 to 13 g, about 7 to 10 g, about 9 to 11 g, or about 10 to 13g. In one embodiment, the amount of collagen present in a 50 g serving is about 10 g. Collagen is a protein and may be present in any available form in the composition. However, the present invention preferably uses collagen peptides. Collagen peptides may be sourced from any known source, including but not limited to, beef, chicken, fish, eggshell membrane, and chicken egg whites. In one preferred embodiment, marine based collagen, such as from white fish is used.

Almond protein blend is present in the composition in an amount of about 25 to 40 weight percent of the composition, about 27 to 32 weight percent of the composition, about 30 to 35 weight percent of the composition, or about 32 to 40 weight percent of the composition. In another embodiment, the amount of almond protein blend present in a 50 g serving is about 12 to 20 g, about 12 to 16 g, about 14 to 18 g, or about 16 g to 20 g. In one embodiment, the amount of almond protein blend present in a 50 g serving is about 16-18 g. Almond protein blends are commercially available and known in the art. The almond protein blend of the present composition is 100% derived from almonds and delivers fat and protein. In the alternative, the present composition may include other nuts in combination with or in lieu of almonds.

The present invention is further directed to a shelf-stable and soft textured functional food composition comprising collagen peptides and almond protein blend that does not include ingredients contain gluten. In one example, forms of sugar are added to the composition that not metabolized by the body, do not cause a high glycemic index response in the blood, and/or do not require labeling as an added sugar on a nutritional label. Such non-sugar sweeteners can be artificial or natural products, including but not limited to the FDA approved products stevia, luo han guo (monk) fruit extract, sucralose, aspartame, acesulfame potassium, neotame, saccharin, advantame, allulose, and/or sugar alcohols extracted from fruit, vegetables, or those that are man-made may be added to the composition of the present invention, including but not limited to erythritol.

In one embodiment of the present invention, the composition may lack any gluten-containing ingredients, i.e., be gluten free, without gluten, or have no gluten.

The composition of the present invention may be in the form of a solid or semi-solid foodstuff, including but not limited to a bar, cracker, or cookie. In one embodiment of the present invention, the composition is formed into a soft bar with a water activity of 0.84 or less.

The present invention is not limited by the method of manufacture. Different types of bar processing equipment (rotary mold, extruder, and cold slab) may be used, though it is known that physically forming the semi-solid or solids of the present invention may negatively affect its texture and chewability, resulting in excessively hard and/or chewy bars. In one preferred embodiment, soft, chewable bars can be made by using the known technique of cold slab manufacturing with suitable pressure. Cold slab manufacturing involves pressing the mixture across a slab, rolling to desired dimensions, and cutting the product at prescribed intervals. The cut product is then placed into cooled baking pans, which can be further cut prior to packaging.

In accordance with the present invention, Tables 1 describes components of suitable compositions:

TABLE 1
50 g serving size: Nutritional information on bar containing, among other
components, collagen with almond protein blend combined with allulose.

| | | Chocolate | Peanut | Mint | | |
|---|---|---|---|---|---|---|
| | Mixed | Almond Sea | Butter | Chocolate | Lemon | Raspberry |
| Parameter | Berry | Salt | Chocolate | Chip | Vanilla | Lemon |
| Calories | 150 | 170 | 160 | 170 | 170 | 150 |
| Total Fat (g) | 7 | 8 | 8 | 9 | 9 | 7 |
| Sat Fat (g) | 2.5 | 3.5 | 4 | 3.5 | 3.5 | 2.5 |
| Trans Fat (g | 0 | 0 | 0 | 0 | 0 | 0 |
| Cholesterol (mg) | 0 | 0 | 0 | 0 | 0 | 0 |
| Sodium (mg) | 160 | 220 | 130 | 180 | 135 | 100 |
| Total | 22 | 19 | 20 | 19 | 18 | 22 |
| Carbohydrate (g) | | | | | | |
| Dietary Fiber (g) | 6 | 5 | 5 | 5 | 5 | 7 |
| Total Sugars (g) | 3 | 2 | 3 | 2 | 1 | 2 |
| Added Sugars (g) | 0 | 2 | 2 | 2 | 0 | 0 |
| Protein (g) | 13 | 14 | 14 | 14 | 14 | 13 |
| Vitamin D (% DV) | 0 | 0 | 0 | 0 | 0 | 0 |
| Calcium (% DV) | 0 | 0 | 0 | 0 | 0 | 0 |
| Iron (% DV) | 8 | 9 | 13 | 8 | 8 | 7 |
| Potassium (% DV) | 5 | 5 | 5 | 4 | 4 | 5 |

In an embodiment of the present invention, the caloric intake of the functional food is less than about 250 calories, less than 200 calories, less than about 150 calories, about 125 calories, or about 100 calories per serving. In one embodiment, the amount of total sugars in the composition is desirably low. For example, the total sugars may be present in an amount of than 6 grams/serving, less than 5 grams/serving, about 5 grams/serving, about 4 grams/serving, about 3 grams/serving, about 2 grams/serving, or about 1 grams/serving. As noted above, the content of the protein, provided at least in part in the form of collagen or collagen peptides, is high, preferably above 10 grams/serving, about 13 grams/serving, or about 14 grams/serving In another embodiment of the invention, no artificial sweeteners or sugars are added to the composition.

### EXAMPLE 1

A composition of the formula depicted in Table 1 was prepared.

1. A functional food composition comprising collagen in an amount of about 15 to 25 weight percent of the composition and nut protein blend in an amount of about 20 to 40 weight percent of the composition.

2. The functional food composition of claim 1, wherein the collagen is present in an amount of about 18 to 22 weight percent of the composition.

3. The functional food composition of claim 1 wherein the collagen is present in the form of collagen peptides.

4. The functional food composition according to claim 1, wherein the nut protein blend is present in an amount of about 33 to 38 weight percent of the composition.

5. The functional food composition according to claim 1, wherein the collagen is present in an amount of about 18 to 22 weight percent of the composition and the nut protein blend is present in an amount of about 33 to 38 weight percent of the composition.

6. The functional food composition according to claim 1, wherein the nut protein blend is selected from the group consisting of almond protein blend, walnut protein blend, coconut protein blend, cashew protein blend, pistachio protein blend, pecan protein blend, macadamia nut protein blend, and brazil nut protein blend.

7. The functional food composition of claim 6, wherein the nut protein blend is almond protein blend.

8. The functional food composition according to claim 1, wherein the composition is a semi-solid or solid.

9. The functional food composition according to claim 1, wherein the composition further comprises one or more non-sugar sweeteners.

10. The functional food composition according to claim 1, wherein the composition comprises less than 20 ppm gluten.

11-12. (canceled)

13. A method of manufacturing a functional food composition comprising mixing collagen in an amount of about 18 to 22 weight percent of the composition and almond protein blend in an amount of about 33 to 38 weight percent of the composition, adding and pressing said mixture to a slab, and cutting said mixture at predetermined locations.
